(19) United States
(12) Patent Application Publication
(10) Pub. No.: US 2025/0262394 A1
(43) Pub. Date: Aug. 21, 2025

(54) SMART INHALERS AND METHODS AND SYSTEMS THEREOF

(71) Applicant: Dilshan Rajan, Plymouth, MN (US)

(72) Inventor: Dilshan Rajan, Plymouth, MN (US)

(21) Appl. No.: 18/582,105

(22) Filed: Feb. 20, 2024

(51) Int. Cl. A61M 15/00; A61M 16/00

(52) U.S. Cl. CPC A61M 15/009; A61M 15/0098; A61M 16/0003; A61M 2016/003; A61M 2205/3331; A61M 2205/3553; A61M 2205/52

(57) ABSTRACT

Disclosed herein are apparatuses for monitoring user health via an inhaler as well as systems and methods thereof. The apparatus can include the inhaler as well as a power source, sensors, and a computing system. The sensors can include a first sensor configured to capture expiratory flow data from within the inhaler and a second sensor configured to capture pulse oximetry or heart rate data from a user of the inhaler. The computing system can be configured to process the data captured by the sensors and record the data as corresponding peak expiratory flow information and pulse oximetry or heart rate information. The apparatus can also include an electronics board, configured to communicatively couple the power source, the sensors, and the computing system.

### TECHNICAL FIELD

The present disclosure relates to inhalers and systems and methods thereof.

### BACKGROUND

An inhaler is a device that provides medicine to the lungs or trachea of a person by the person inhaling the medicine when dispensed by the device. The inhalation of the medicine dispensed by the device allows the medicine to be absorbed by tissue proximate to or within the lungs, which provides targeted delivery of the medicine to a specific region while reducing the side effects of orally taken medications. There are many types of inhalers to treat various conditions such as asthma, chronic obstructive pulmonary disease (COPD), and other diseases and disorders related to a person's airways.

### SUMMARY

Disclosed herein are smart inhalers. In other words, disclosed herein are variations on an inhaler that include at least part of a computing system as well as one or more sensors to capture, process, and provide information about a user using the inhaler. The inhaler and variations of the inhaler can provide feedback information from its use—such as feedback including flow data related to a user's breathing, pulse oximetry data of the user, heart rate data of the user, and other types of feedback information related to a condition or health status of the user. Such information in the form of feedback from the inhaler can be used to assess a disease or disorder experienced by the user, such as asthma.

Also, disclosed herein are apparatuses for monitoring user health via an inhaler as well as systems and methods thereof. Such an apparatus can include the inhaler as well as a power source, sensors, and a computing system. The sensors can include a first sensor configured to capture expiratory flow data from within the inhaler and a second sensor configured to capture pulse oximetry or heart rate data from a user of the inhaler. The computing system can be configured to process the data captured by the sensors and record the data as corresponding peak expiratory flow information and pulse oximetry or heart rate information. The apparatus can also include an electronics board, configured to communicatively couple the power source, the sensors, and the computing system. Such a method or system can include such an apparatus in some embodiments.

The inhalers shown in the drawings are metered-dose inhalers (MDIs); however, it is to be understood that the inhalers of some embodiments are dry powder inhalers (DPIs) or Soft mist inhalers (SMIs). Also, in some cases, embodiments can include a nebulizer.

These and other important aspects of the invention are described more fully in the detailed description below. The invention is not limited to the particular assemblies, apparatuses, methods, and systems disclosed herein. Other embodiments can be used and changes to the described embodiments can be made without departing from the scope of the claims that follow the detailed description.

### BRIEF DESCRIPTION OF THE DRAWINGS

The present disclosure will be understood more fully from the detailed description given below and from the accompanying drawings of various example embodiments of the disclosure.

FIGS. 1 to 5 illustrate different views of an example inhaler, in accordance with some embodiments of the present disclosure. In the views, some portions of the inhaler are broken away to reveal internal details of the construction of the inhaler.

FIG. 6 illustrates a block diagram of example aspects of a computing system, which can include computing components, sensors, and a communications interface of the inhaler shown in FIGS. 1 to 5, in accordance with some embodiments of the present disclosure.

FIG. 7 shows a method related to an example inhaler (such as the inhaler shown in FIGS. 1 to 5) and an example computing system (such as the computing system shown in FIG. 6), in accordance with some embodiments of the present disclosure.

FIG. 8 illustrates a second example inhaler in which the flow rate information and other health-related information are captured and processed by a separate attachable system, in accordance with some embodiments of the present disclosure.

### DETAILED DESCRIPTION

The present disclosure will be understood more fully from the detailed description given below and from the accompanying drawings of various example embodiments of the disclosure.

Disclosed herein are smart inhalers. In other words, disclosed herein are variations on an inhaler that include at least part of a computing system as well as one or more sensors to capture, process, and provide information about a user using the inhaler. The inhaler and variations of the inhaler can provide feedback information from its use—such as feedback including flow data related to a user's breathing, pulse oximetry data of the user, heart rate data of the user, and other types of feedback information related to a condition or health status of the user. Such information in the form of feedback from the inhaler can be used to assess a disease or disorder experienced by the user, such as asthma.

Also, disclosed herein are apparatuses for monitoring user health via an inhaler as well as systems and methods thereof. Such an apparatus can include the inhaler as well as a power source, sensors, and a computing system. The sensors can include a first sensor configured to capture expiratory flow data from within the inhaler and a second sensor configured to capture pulse oximetry or heart rate data from a user of the inhaler. The computing system can be configured to process the data captured by the sensors and record the data as corresponding peak expiratory flow information and pulse oximetry or heart rate information. The apparatus can also include an electronics board, configured to communicatively couple the power source, the sensors, and the computing system. Such a method or system can include such an apparatus in some embodiments.

The inhalers shown in the drawings are metered-dose inhalers (MDIs); however, it is to be understood that the inhalers of some embodiments are dry powder inhalers (DPIs) or Soft mist inhalers (SMIs). Also, in some cases, embodiments can include a nebulizer.

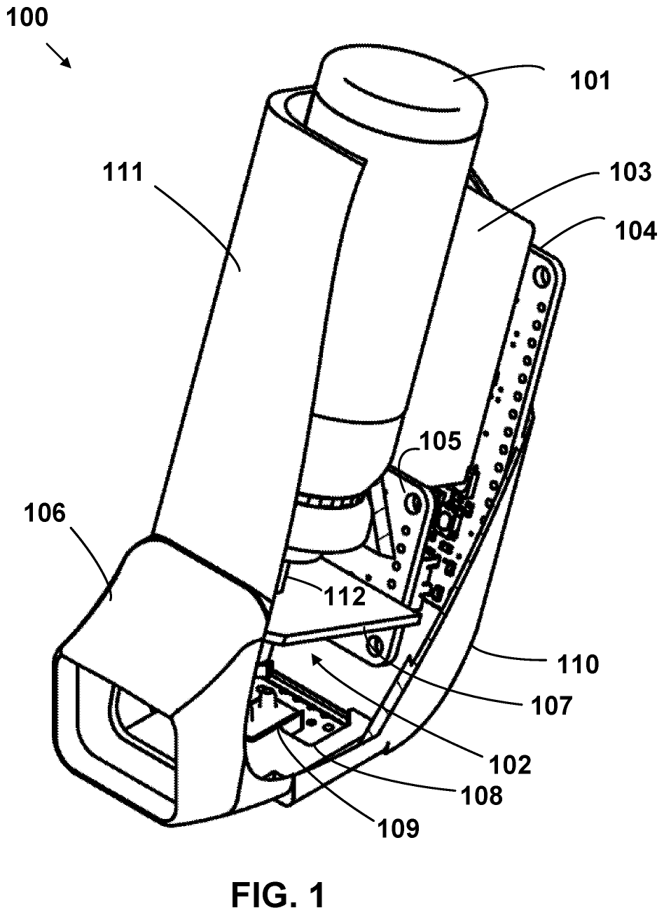

FIGS. 1 to 5 illustrate different views of an example inhaler 100, in accordance with some embodiments of the present disclosure. In the views, some portions of the inhaler 100 are broken away to reveal internal details of the construction of the inhaler.

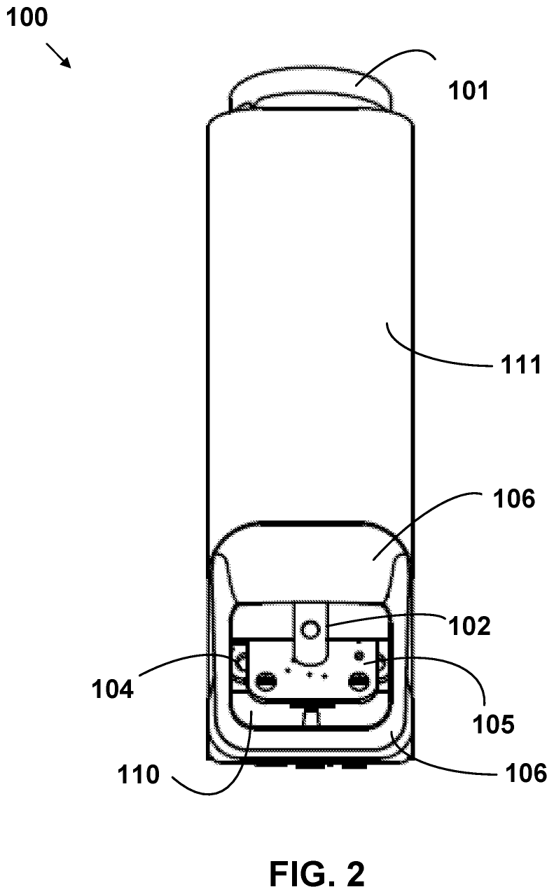

FIG. 1 shows a front-side perspective view of the inhaler 100 with broken-away portions of the inhaler. For example, parts of the housing and actuator (such as a plastic actuator) are broken away to reveal an inhaler medication cartridge 101 (also referred to herein as a canister) and the spray nozzle 112 of the cartridge. The medication aerosolizer 102 is not depicted in FIG. 1, but an arrow is pointing to a position under a spray nozzle plate 107, which is where the aerosolizer is located (e.g., see FIGS. 2, 3, and 5 for a depiction of the aerosolizer 102). Also, revealed is a power source of the inhaler 100. And, in the illustration, battery 103 is shown as the power source. Also revealed is a microcontroller 104 which can include or be a part of some of the computing systems described herein. Also revealed is a first integrated circuit 105 that can include an accelerometer, gyroscope, or a combination thereof. The inhaler 100 also includes a mouthpiece 106. As revealed by the breakaway of the housing of the inhaler 100, the inhaler also includes the spray nozzle plate 107. The plate 107 causes the spray nozzle 112 to spray medicine contained in the cartridge 101 when the actuator is pushed downward by a user pressing the front end of the canister on the plate 107. The spray nozzle 107 releases the medicine from the cartridge 101 and the medicine is aerosolized by the medication aerosolizer 102. Also, FIG. 1 reveals a second integrated circuit 108 that can include a pulse oximeter, a heart rate sensor, or a combination thereof. Furthermore, FIG. 1 reveals a differential pressure sensor 109. FIG. 1 also shows electronics housing 110 and main body housing 111. As depicted, the electronic housing 110 can house the battery 103, the microcontroller 104, the first integrated circuit 105, and the second integrated circuit 108. In some examples, the electronics housing houses the differential pressure sensor 109 too. In some other examples, the main body housing 111 houses the differential pressure sensor 109. The cartridge 101 fits into a reusable medication cartridge holder and interface which is a part of the main body housing 111 of the inhaler 100. The actuator of the inhaler 100 includes the main body housing 111.

FIG. 2 shows a front perspective view of the inhaler 100 such that the medication aerosolizer 102, the microcontroller 104, and the first integrated circuit 105 are shown through the opening provided by the mouthpiece 106. Also, FIG. 2 illustrates a top portion of the inhaler medication cartridge 101 and a front portion of an external wall of the main body housing 111. As shown, the medication aerosolizer 102 is in front of the first integrated circuit 105, which is in front of the microcontroller 104. Through the opening of the mouthpiece 106, an internal wall of the electronics housing 110 is shown behind the medication aerosolizer 102, the first integrated circuit 105, and the microcontroller 104.

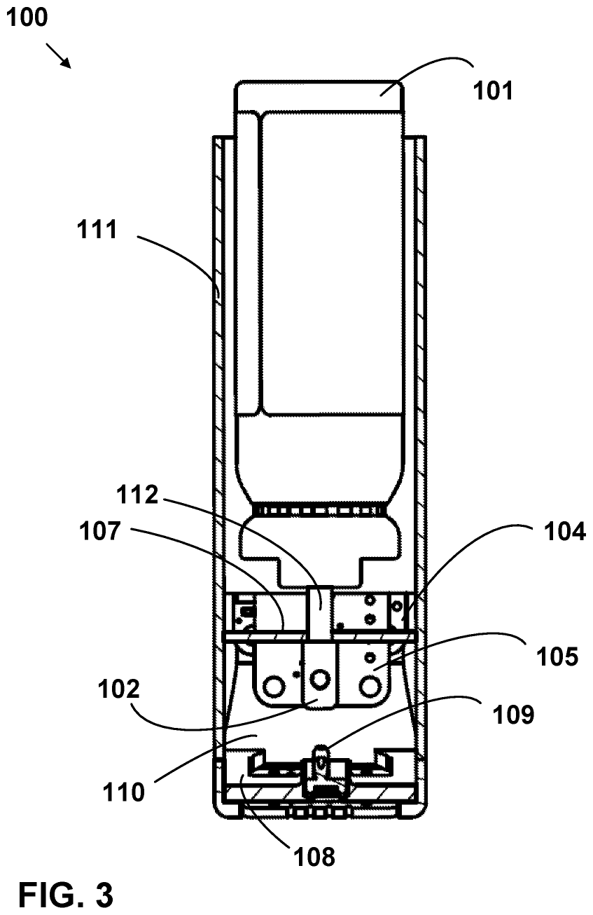

FIG. 3 shows a front and cross-sectional perspective view of the inhaler 100 with broken-away portions of the inhaler. For example, a front portion of the housing 111 and actuator (such as a plastic actuator) are broken away to reveal an entire front-side portion of the inhaler medication cartridge 101, the spray nozzle 112, and the medication aerosolizer 102. As shown the nozzle 112 interfaces the aerosolizer 102 via an interface portion of a reusable medication cartridge holder of the inhaler 100. By having the parts broken away, FIG. 3 also reveals a front view of the aerosolizer 102, the microcontroller 104, the first integrated circuit 105, the spray nozzle plate 107, the second integrated circuit 108, the differential pressure sensor 109, and the spray nozzle 112. An internal wall of the electronics housing 110 is also shown behind the medication aerosolizer 102, the first integrated circuit 105, and the microcontroller 104.

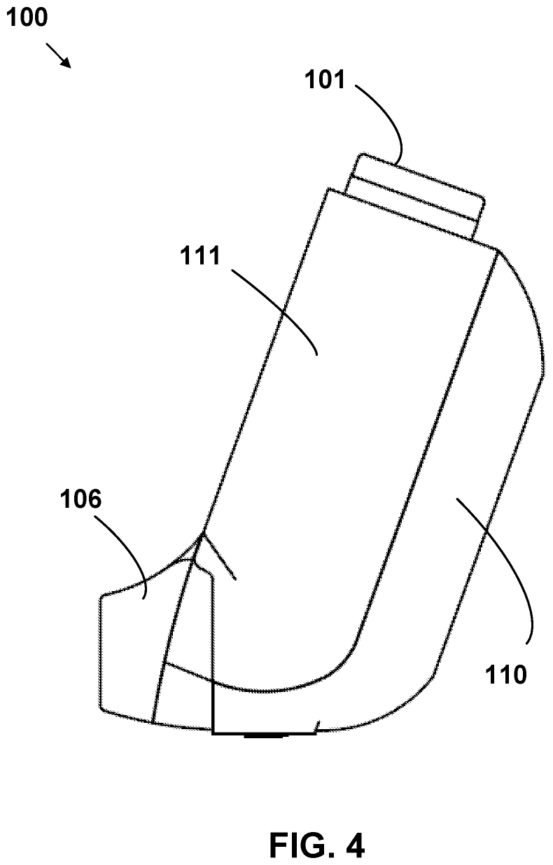

FIG. 4 shows a side perspective view of the inhaler 100. FIG. 4 depicts external walls of respective side portions of the mouthpiece 106, the electronics housing 110, and the main body housing 111. Also, a top portion of the inhaler medication cartridge 101 is shown.

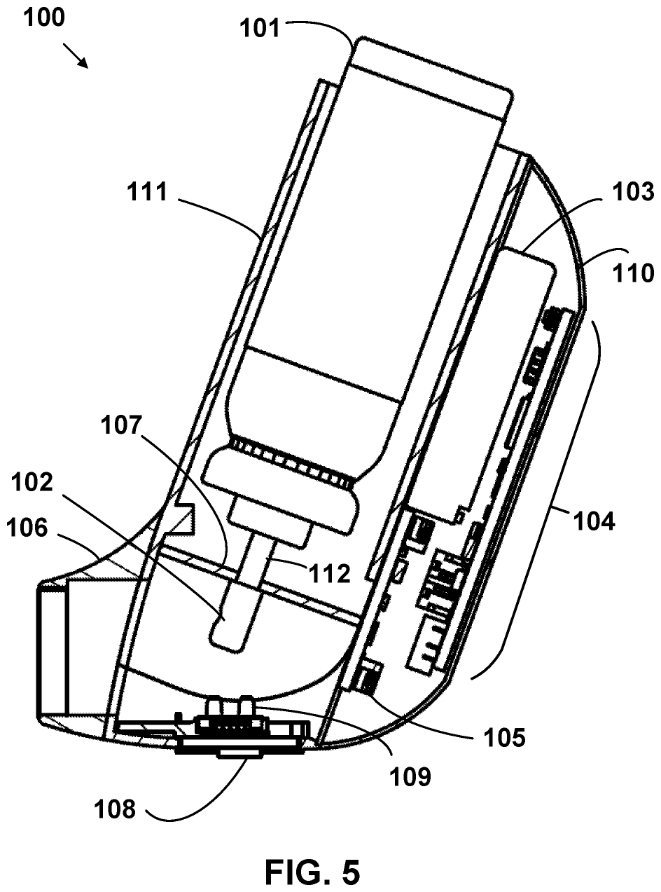

FIG. 5 shows a side and cross-sectional perspective view of the inhaler 100 with broken-away portions of the inhaler. For example, respective side portions of the main body housing 111 and the electronics housing 110 as well as the actuator (such as a plastic actuator) are broken away to reveal portions of the inhaler medication cartridge 101, the spray nozzle 112 attached to the cartridge, and medication aerosolizer 102, the battery 103, the microcontroller 104, the first integrated circuit 105, the spray nozzle plate 107, and the differential pressure sensor 109. Also, shown is a part extending from the second integrated circuit 108 (which can include a pulse oximeter, a heart rate sensor, or a combination thereof) that is configured to be pressed upon by a thumb of a user when the user holds and activates the inhaler 100. Wherein the circuit 108 includes a pulse oximeter, the pulse oximeter can capture and measure, such with a computing system, the saturation of oxygen carried in red blood cells of a user. The capturing of the saturation of oxygen can occur via the thumb of the user pressed upon the part extending from the second integrated circuit 108.

As shown by FIGS. 1 to 5, some embodiments of an apparatus can include an inhaler (e.g., see inhaler 100) that includes a plurality of inhaler components including a power source (e.g., see battery 103), a plurality of sensors including a first sensor configured to capture expiratory flow data from the inhaler (e.g., see the differential pressure sensor 109 as well as such as via the opening in the mouthpiece 106) as well a second sensor configured to capture pulse oximetry or heart rate data from a user of the inhaler (e.g., see circuit 108), and a computing system (e.g., see microcontroller 104) configured to process the data captured by the plurality of sensors and record the data as corresponding peak expiratory flow information and pulse oximetry or heart rate information. Also, the inhaler can include an electronics board, configured to communicatively couple the power source, plurality of sensors, and the computing system (e.g., see microcontroller 104, first circuit 105, and second circuit 108).

In some embodiments of the apparatus, the plurality of inhaler components include a reusable-medication-cartridge holder and interface (e.g., see cartridge 101, housing 111, and plate 107), wherein the holder is configured to secure a medication cartridge within the inhaler housing, and wherein the interface is configured to connect the medication cartridge with a hand-operated actuator and a metering valve of the inhaler (e.g., see medication aerosolizer 102 which can include a metering valve in some embodiments). In some embodiments of the apparatus, the metering valve is a part of or in electromechanical communication with the first sensor. In some embodiments of the apparatus, the second sensor is configured to capture the pulse oximetry or heart rate data via a thumb of a user of the inhaler when the user holds the inhaler to activate the hand-operated actuator of the inhaler to activate the inhaler and release medication from the inhaler (e.g., see the part extending from the circuit 108). In some embodiments of the apparatus, the inhaler includes a pressurized metered-dose inhaler (MDI) including the actuator and the metering valve and the medication cartridge includes a metal canister and medication within the canister. In some examples, the medication includes a propellant or suspension and the actuator and the metering valve are configured to be reused with replacements of the medication cartridge. In some cases, the actuator is configured to receive and attach to the canister. In some cases, the apparatus includes an aerosolizer nozzle and plate (e.g., see nozzle 112 and plate 107) configured to release and spray medication from the medication cartridge, and beneath a base of the nozzle and plate a part of the first sensor (such as a digital differential pressure sensor, e.g., see sensor 109) is pointed upward and a part of the second sensor (such as a pulse oximeter) is pointed downward such that a thumb of a user is placed underneath the second sensor and on a data capturing portion of the second sensor (e.g., see the part extending from circuit 108 shown in FIG. 5).

In some examples of the apparatus, the first sensor includes a peak flow meter. The first sensor can include a digital differential pressure sensor to measure the peak expiratory flow (e.g., see sensor 109). In some examples of the apparatus, the second sensor is configured to capture the pulse oximetry or heart rate data via a thumb of a user of the inhaler when the user holds the inhaler to activate a hand-operated actuator of the inhaler to activate the inhaler and release medication from the inhaler (e.g., see the part extending from circuit 108 shown in FIG. 5). In some cases, the second sensor includes pulse oximeter.

In some examples of the apparatus, the plurality of sensors includes a motion sensor configured to detect movement of the apparatus and the motion sensor includes a gyroscope or an accelerometer (e.g., see the first integrated circuit 105). In some examples of the apparatus, the power source is communicatively coupled to a charging system, and the charging system is connected to the electronics board (e.g., see battery 103 which can be communicatively coupled to the microcontroller 104, which in some cases includes a charging system). In some examples of the apparatus, the electronics board includes a printed circuit board (PCB).

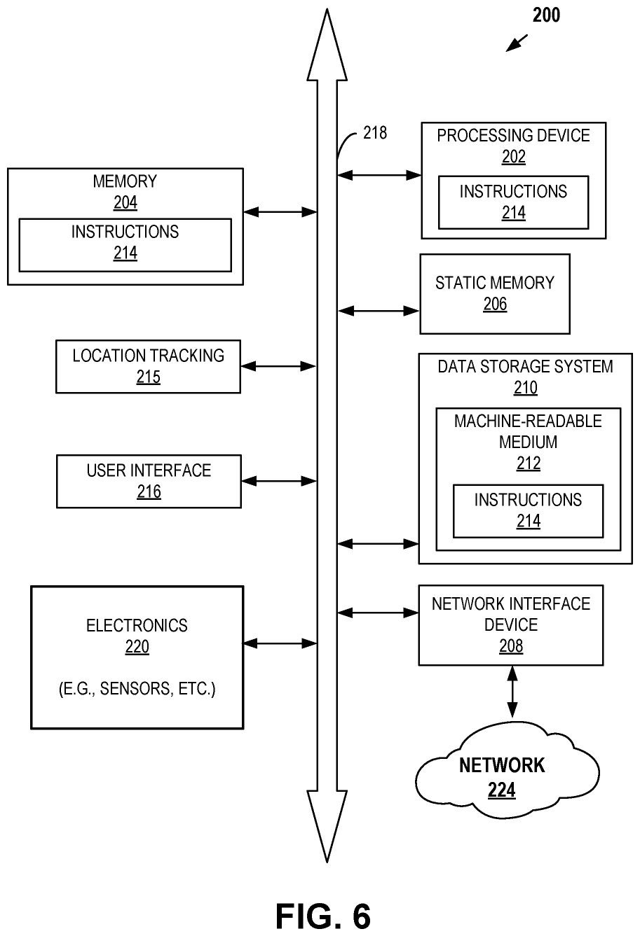

FIG. 6 illustrates a block diagram of example aspects of a computing system 200, which can include computing components, sensors, and a communications interface of the inhaler shown in FIGS. 1 to 5, in accordance with some embodiments of the present disclosure. Also, FIG. 5 illustrates parts of the computing system 200 within which a set of instructions are executed for causing a machine (such as a computer processor or processing device 202) to perform any one or more of the steps of the methodologies discussed herein performed by a computing system (e.g., see the method steps of the method 300 shown in FIG. 6). In some embodiments, the computing system 200 operates with additional computing systems to provide increased computing capacity in which multiple computing systems operate together to perform any one or more of the methodologies or processes discussed herein that are performed by a computing system.

In some embodiments, the computing system 200 corresponds to a host system that includes, is coupled to, or utilizes memory or is used to perform the operations performed by any one of the computing systems described herein. In some embodiments, the machine is connected (e.g., networked) to other machines in a LAN, an intranet, an extranet, or the Internet. In some embodiments, the machine operates in the capacity of a client device in a client-server network environment, as a peer machine in a peer-to-peer (or distributed) network environment, or as a client in a cloud computing infrastructure or environment. In some embodiments, the machine is a computer or microcomputer or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies or processes discussed herein performed by computing systems.

The computing system 200 includes a processing device 202, a main memory 204 (e.g., read-only memory (ROM), flash memory, dynamic random-access memory (DRAM), etc.), a static memory 206 (e.g., flash memory, static random-access memory (SRAM), etc.), and a data storage system 210, which communicate with each other via a bus 218. The processing device 202 represents one or more general-purpose processing devices such as a microprocessor, a central processing unit, or the like. More particularly, the processing device can include a microprocessor or a processor implementing other instruction sets, or processors implementing a combination of instruction sets. Or, the processing device 202 is one or more special-purpose processing devices such as an application-specific integrated circuit (ASIC), a field programmable gate array (FPGA), a digital signal processor (DSP), a network processor, or the like. The processing device 202 is configured to execute instructions 214 for performing the operations discussed herein performed by a computing system. In some embodiments, the computing system 200 includes a network interface device 208 to communicate over a communications network such as network 224. Such a communications network can include one or more local area networks (LAN(s)), such as a WIFI network or a BLUETOOTH network, and/or one or more wide area networks (WAN(s)). In some embodiments, the communications network includes the Internet and/or any other type of interconnected communications network. The communications network can also include a single computer network or a telecommunications network. In the case of communications network 224 including the Internet, the inhaler 100 or another embodiment of the inhaler can be considered Internet of Things (IoT) device.

The data storage system 210 includes a machine-readable storage medium 212 (also known as a computer-readable medium) on which is stored one or more sets of instructions 214 or software embodying any one or more of the methodologies or functions described herein performed by a computing system. The instructions 214 also reside, completely or at least partially, within the main memory 204 or within the processing device 202 during execution thereof by the computing system 200, the main memory 204 and the processing device 202 also constituting machine-readable storage media. While the machine-readable storage medium 212 is shown in an example embodiment to be a single medium, the term “machine-readable storage medium” should be taken to include a single medium or multiple media that store the one or more sets of instructions. The term “machine-readable storage medium” shall also be taken to include any medium that is capable of storing or encoding a set of instructions for execution by the machine and that causes the machine to perform any one or more of the methodologies of the present disclosure performed by a computing system. The term “machine-readable storage medium” shall accordingly be taken to include solid-state memories, optical media, or magnetic media.

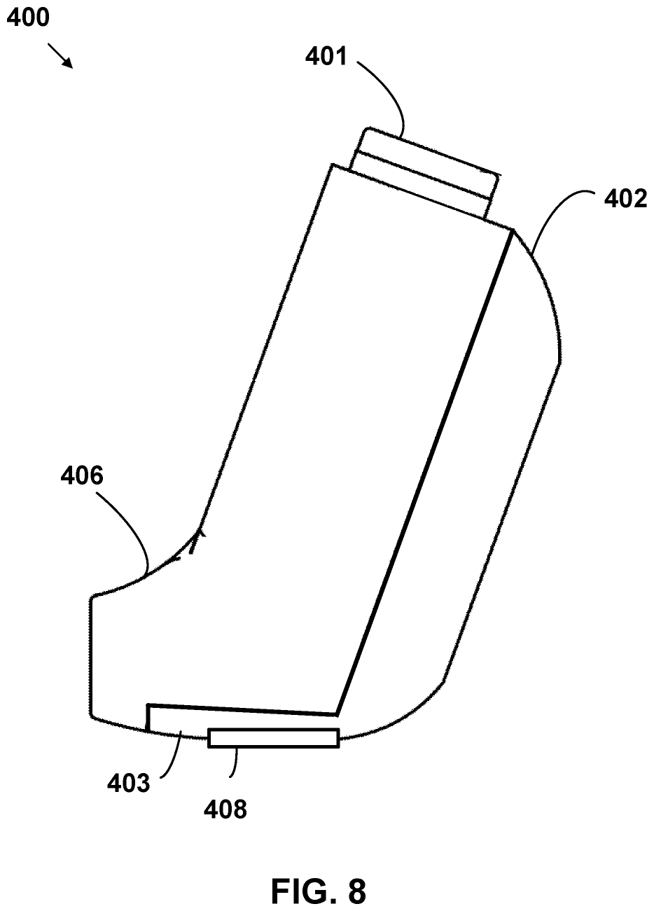

Also, as shown, the computing system 200 includes a user interface 216. A UI, such as UI 216, or a UI device described herein includes any space or equipment where interactions between humans and machines occur. A UI described herein allows the operation and control of the machine from a human user, while the machine simultaneously provides feedback information to the user. Examples of a user interface, or UI device include the interactive aspects of computer operating systems (such as GUIs) and inhaler operator controls such as the actuator of an inhaler (e.g., the plastic actuator on an MDI). Also, as shown, the computing system 200 includes electronics 220 that are a part of the computing system or interact directly with the computing system such as any one of the sensors described herein (such as the sensor of circuit 108 shown in FIG. 1 and sensor 408 shown in FIG. 8.

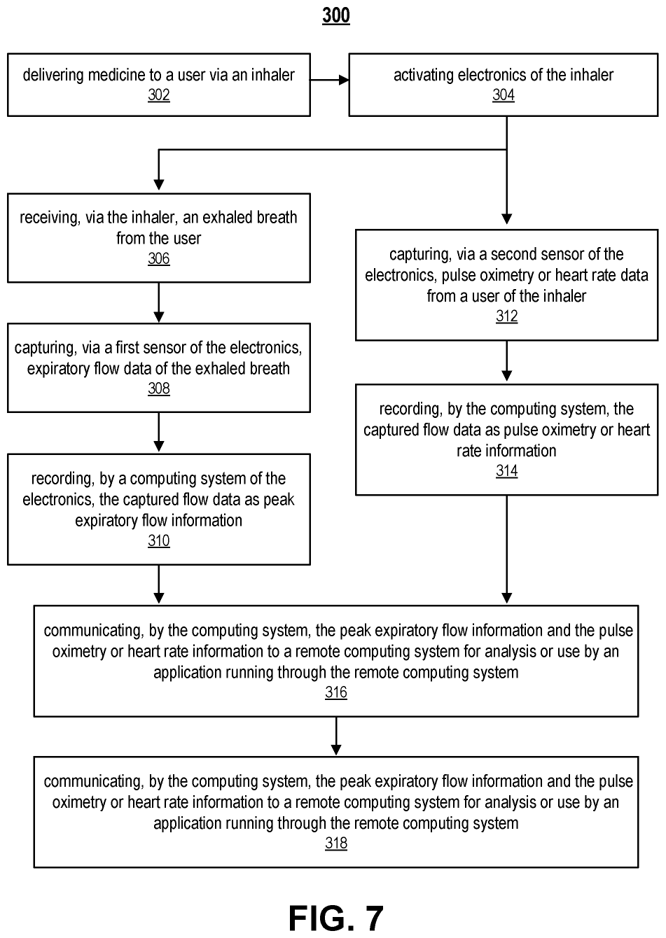

FIG. 7 shows a method 300 related to an example inhaler (such as the inhaler 100 shown in FIGS. 1 to 5 and FIG. 8) and an example computing system (such as the computing system 200 shown in FIG. 6), in accordance with some embodiments of the present disclosure. The method 300 starts with delivering medicine to a user via an inhaler at step 302. At step 304, the method continues with activating the electronics of the inhaler by use or motion of the inhaler by the user sensed by a motion sensor of the inhaler. At step 306, the method continues with receiving, via the inhaler, an exhaled breath from the user. At step 308, the method continues with capturing, via a first sensor of the electronics, expiratory flow data of the exhaled breath. In some cases, the first sensor is located within the inhaler and beneath an aerosolizer nozzle of the inhaler. At step 310, the method continues with recording, by a computing system of the electronics, the captured flow data as peak expiratory flow information. At step 312, the method continues with capturing, via a second sensor of the electronics that is integrated with the inhaler, pulse oximetry or heart rate data from a user of the inhaler. At step 314, the method continues with recording, by the computing system, the captured flow data as pulse oximetry or heart rate information.

At step 316, the method continues with communicating, by the computing system, the peak expiratory flow information and the pulse oximetry or heart rate information to a remote computing system for analysis or use by an application running through the remote computing system. At step 318, the method continues with the application generating and providing health information via a user interface based on the peak expiratory flow information and the pulse oximetry or heart rate information. In some cases, the method further includes the application providing a periodic or daily message to the user via the user interface based at least partially on the peak expiratory flow information and the pulse oximetry or heart rate information.

In some cases of the method, the second sensor captures the pulse oximetry or heart rate data via a thumb of a user of the inhaler when the user holds the inhaler to activate a hand-operated actuator of the inhaler to activate the inhaler and release medication from the inhaler. And, in some examples, the method includes the second sensor capturing the pulse oximetry or heart rate data via a thumb of a user of the inhaler when the user holds the inhaler after breathing into the inhaler to measure expiratory flow data.

FIG. 8 illustrates a second example inhaler 400 in which the flow rate and other health-related information is captured and processed by a separate attachable system (see the clip-on system 402). As shown in FIG. 8, some embodiments include an apparatus that includes an inhaler (such as inhaler 400), in which the inhaler includes a plurality of inhaler components (such as medicine cartridge 401) as well as a separate attachable system (see clip-on system 402), configured to removably attach to the inhaler. Similarly, the inhaler components also include a mouthpiece 406. The separate attachable system includes a power source, a plurality of sensors, and a computing system. The sensors include at least a first sensor configured to capture expiratory flow data from the inhaler as well as a second sensor configured to capture pulse oximetry or heart rate data from a user of the inhaler (e.g., see the thumb sensor 408 at the lower part 403 of the clip-on system 402). The computing system is configured to process the data captured by the plurality of sensors and record the data as corresponding peak expiratory flow information and pulse oximetry or heart rate information. The separate attachable system also includes an electronics board, configured to communicatively couple the power source, plurality of sensors, and the computing system. In summary, the inhaler 400 has similar components to the inhaler 100 but the electronics of the inhaler 400 are part of a separate module (see clip-on system 402). As the name implies, the clip-on system 402 clips onto the outside wall of an inhaler or the actuator of an inhaler.

An example problem faced in asthma control is that asthma levels are assessed once or a couple of times a year, making it very difficult to assess the status of asthma in a patient. And, this is especially a problem since asthma can change seasonally. As a result, many people become ill enough to be taken to the emergency room or they become disabled further with pneumonia (which could lead to hospitalization). This can occur because of a false sense of security in thinking their asthma is well controlled when in actuality it is not. The apparatuses, inhalers, and system and methods thereof described herein address these problems by having a sensor built in (such as a digital differential pressure sensor, e.g., see sensor 109). Such a sensor can capture daily peak flow data. Also, a second sensor (e.g., see circuit 108 or thumb sensor 408) can capture pulse oximetry or heart rate, both of which can be used to assess the state of asthma of a user. This way health status is known more readily by the patient and the doctor of the patient potentially and action can be taken more immediately to prevent hospital and emergency room visits as well as further disease that can occur from neglect. Another major problem is that current inhalers are not reusable. In some embodiments, the apparatuses, inhalers, and systems and methods thereof described herein are reusable. For example, medicine cartridges can be replaced while keeping the body of the inhaler intact for another cartridge.

In some examples, the inhaler or apparatus including the inhaler uses a relatively normal inhaler body shape with some modifications. Also, the inhaler or apparatus can include an electronic board, gyroscope, and battery in a chamber behind the main chamber of the inhaler. Such an apparatus can also have a pulse oximeter placed under the thumb grip of the inhaler, and a digital differential pressure sensor to measure the peak expiratory flow.

In some examples, a pulse oximeter, heart rate monitor, or combination thereof and a differential flow meter are in the inhaler body, as well as a gyroscope or accelerometer, a PCB, and a rechargeable battery can be part of the inhaler. When the inhaler is used (such as daily), after daily medication, the smart features would be awakened via the gyroscope (e.g., shake to awake), and a user can blow into the inhaler, which would record peak flow data via the digital differential pressure sensor and collect pulse oximetry and heart rate data from a sensor under the thumb rest area where the user would grip the inhaler naturally during use (e.g., see method 300). Such captured data can then be transmitted to an application, where trends can be analyzed to view how controlled and severe the asthma is (e.g., see method 300). The application can also send daily reminders to a user's phone to remind the user of the inhaler. In some cases, as mentioned, the inhaler body is reusable, and when the medication cartridge is empty, the user can remove the previous cartridge, and insert a new one. In some examples, the electronics of the apparatus are designed so that none of the integrated sensors affect the function of the inhaler medication administering system. In some cases, the disclosed technologies aim to keep them as similar to a regular inhaler as possible to limit the change in patients' workflow or routine for using inhalers. With that in mind, the sensors can be built into the inhaler body. After the patient takes their inhaler, they would then blow into the inhaler body, which would collect the peak flow data. The flow sensor is on the inside, under the inhaler nozzle. The pulse oximeter data and heart rate data are recorded from a sensor on the thumb pad under the inhaler, which is where a user would hold it to use the inhaler correctly. As shown as an example in FIG. 8, an alternative system can have a clip-on system that contains the sensors rather than having them built into the inhaler (contrary to the inhaler shown in FIGS. 1 to 5).

In the foregoing specification, embodiments of the disclosure have been described with reference to specific example embodiments thereof. It will be evident that various modifications can be made thereto without departing from the broader spirit and scope of embodiments of the disclosure as set forth in the following claims. The specification and drawings are, accordingly, to be regarded in an illustrative sense rather than a restrictive sense.

What is claimed is:

1. An apparatus, comprising:
an inhaler, comprising a plurality of inhaler components;
power source;
a plurality of sensors, comprising:
a first sensor configured to capture expiratory flow data from the inhaler; and
a second sensor configured to capture pulse oximetry or heart rate data from a user of the inhaler;
a computing system configured to process the data captured by the plurality of sensors and record the data as corresponding peak expiratory flow information and pulse oximetry or heart rate information; and
an electronics board, configured to communicatively couple the power source, plurality of sensors, and the computing system.

2. The apparatus of claim 1, wherein the plurality of inhaler components comprise a reusable-medication-cartridge holder and interface, wherein the holder is configured to secure a medication cartridge within the inhaler housing, and wherein the interface is configured to connect the medication cartridge with a hand-operated actuator and a metering valve of the inhaler.

3. The apparatus of claim 2, wherein the metering valve is a part of or in electromechanical communication with the first sensor.

4. The apparatus of claim 2, wherein the second sensor is configured to capture the pulse oximetry or heart rate data via a thumb of a user of the inhaler when the user holds the inhaler to activate the hand-operated actuator of the inhaler to activate the inhaler and release medication from the inhaler.

5. The apparatus of claim 2,
wherein the inhaler comprises a pressurized metered-dose inhaler comprising the actuator the metering valve,
wherein the medication cartridge comprises a metal canister and medication within the metal canister,
wherein the medication comprises a propellant or suspension,
wherein the actuator and the metering valve are configured to be reused with replacements of the medication cartridge, and
wherein the actuator is configured to receive and attach to the canister.

6. The apparatus of claim 2, comprising an aerosolizer nozzle and plate configured to release and spray medication from the medication cartridge, and beneath a base of the nozzle and plate a part of the first sensor is pointed upward and a part of the second sensor is pointed downward such that a thumb of a user is placed underneath the second sensor and on a data capturing portion of the second sensor.

7. The apparatus of claim 1, wherein the first sensor comprises a peak flow meter.

8. The apparatus of claim 7, wherein the first sensor comprises a digital differential pressure sensor to measure the peak expiratory flow.

9. The apparatus of claim 1, wherein the second sensor is configured to capture the pulse oximetry or heart rate data via a thumb of a user of the inhaler when the user holds the inhaler to activate a hand-operated actuator of the inhaler to activate the inhaler and release medication from the inhaler.

10. The apparatus of claim 1, wherein the second sensor comprises a pulse oximeter.

11. The apparatus of claim 1, wherein the plurality of sensors comprises a motion sensor configured to detect movement of the apparatus, and wherein the motion sensor comprises a gyroscope or an accelerometer.

12. The apparatus of claim 1, wherein the power source is communicatively coupled to a charging system, and wherein the charging system is connected to the electronics board.

13. The apparatus of claim 1, wherein the electronics board comprises a printed circuit board (PCB).

14. A method, comprising:
delivering medicine to a user via an inhaler;
activating electronics of the inhaler by use or motion of the inhaler by the user sensed by a motion sensor of the inhaler;
receiving, via the inhaler, an exhaled breath from the user;
capturing, via a first sensor of the electronics, expiratory flow data of the exhaled breath, wherein the first sensor is located within the inhaler and beneath an aerosolizer nozzle of the inhaler;
recording, by a computing system of the electronics, the captured flow data as peak expiratory flow information;
capturing, via a second sensor of the electronics and integrated with the inhaler, pulse oximetry or heart rate data from a user of the inhaler; and
recording, by the computing system, the captured flow data as pulse oximetry or heart rate information.

15. The method of claim 14, further comprising communicating, by the computing system, the peak expiratory flow information and the pulse oximetry or heart rate information to a remote computing system for analysis or use by an application running through the remote computing system.

16. The method of claim 15, further comprising the application generating and providing health information via a user interface based on the peak expiratory flow information and the pulse oximetry or heart rate information.

17. The method of claim 16, further comprising the application providing a periodic or daily message to the user via the user interface based at least partially on the peak expiratory flow information and the pulse oximetry or heart rate information.

18. The method of claim 14, wherein the second sensor captures the pulse oximetry or heart rate data via a thumb of a user of the inhaler when the user holds the inhaler to activate a hand-operated actuator of the inhaler to activate the inhaler and release medication from the inhaler.

19. The method of claim 14, wherein the second sensor captures the pulse oximetry or heart rate data via a thumb of a user of the inhaler when the user holds the inhaler after breathing into the inhaler to measure expiratory flow data.

20. An apparatus, comprising:
an inhaler, comprising a plurality of inhaler components; and
a separate attachable system, configured to removably attach to the inhaler and comprising:
power source;
a plurality of sensors, comprising:
a first sensor configured to capture expiratory flow data from the inhaler; and
a second sensor configured to capture pulse oximetry or heart rate data from a user of the inhaler;
a computing system configured to process the data captured by the plurality of sensors and record the data as corresponding peak expiratory flow information and pulse oximetry or heart rate information; and
an electronics board, configured to communicatively couple the power source, plurality of sensors, and the computing system.
